(19) United States
(12) Patent Application Publication
(10) Pub. No.: US 2026/0114730 A1
(43) Pub. Date: Apr. 30, 2026

(54) RETINAL IMAGE CAPTURING

(71) Applicant: MEDIOS TECHNOLOGIES PTE LTD, Singapore (SG)

(72) Inventors: Florian Mickael SAVOY, Estilo (SG); Bhargav SOSALE, Singapore (SG); Chiran Mandula BOPITIYA, Singapore (SG)

(73) Assignee: MEDIOS TECHNOLOGIES PTE LTD, Singapore (SG)

(21) Appl. No.: 18/704,656

(22) PCT Filed: Oct. 5, 2022
(86) PCT No.: PCT/SG2022/050712

§ 371 Date: Apr. 25, 2024

(30) Foreign Application Priority Data
Oct. 27, 2021 (SG) 10202111949W

(51) Int. Cl. A61B 3/14; A61B 3/12

(52) U.S. Cl. CPC A61B 3/14; A61B 3/12

(57) ABSTRACT

RETINAL IMAGE CAPTURING Approaches for automated retinal image capturing are described. In an example, a plurality of target frames recorded by an imaging device are received, wherein the plurality of target frames relate to a retina of a subject. The plurality of target frames are then analyzed based on a trained analysis model. For example, the analysis model is trained based on a set of training images, wherein the analysis model incorporates a set of confidence weights based on visual attributes and annotations of the set of training images. Based on the analysis of the plurality of target frames, an imaging device may be triggered to capture a retinal image of the retina of the subject.

### BACKGROUND

An image capturing device may be used for capturing retinal images for medical examining of an eye. Capturing a retinal image in handheld mode with the image capturing device may be a tedious process. In such cases, the image capturing device may have to be positioned correctly at a correct distance from the eye of a subject. Moreover, the image capturing device may have to point precisely through pupil of the eye for capturing the retinal image. This requires a user of the image capturing device to assess brightness across an environment and accordingly adjust the image capturing device to capture the retinal image.

### BRIEF DESCRIPTION OF DRAWINGS

The following detailed description references the drawings, wherein:

FIG. 1 illustrates an example system for automated retinal image capturing, based on an analysis model, according to an example of the present subject matter;

FIG. 2 illustrates a training system for training an analysis model, according to an example of the present subject matter;

FIGS. 3 and 4 illustrate set of training images for training an analysis model, according to an example of the present subject matter;

FIG. 5 illustrates an assessment system implementing an analysis model, according to an example of the present subject matter;

FIG. 6 illustrates an example method for training an analysis model at a training system, in accordance with example of the present subject matter; and

FIG. 7 illustrates a system environment implementing a non-transitory computer readable medium for automated retinal image capturing, based on an analysis model, in accordance with example of the present subject matter.

Throughout the drawings, identical reference numbers designate similar, but not necessarily identical, elements. The drawings provide examples and/or implementations consistent with the description; however, the description is not limited to the examples and/or implementations provided in the drawings.

### DETAILED DESCRIPTION

An imaging device may be a device capable of capturing and storing still or moving images. In certain cases, the imaging device may also execute image processing to output an image. The imaging device may include a plurality of components for recording, storing, manipulating, viewing and transmitting of visual images. Examples of components of the imaging device may include, but are not limited to, an optical aperture, an imaging lens, a micro lens array, an imaging element, a processor, and a controller. Examples of the imaging device include, but are not limited to, still camera, camcorder, motion picture camera, and 3D camera.

Imaging device may be used for a variety of medical applications. The imaging device may be used for performing imaging of various parts of human body, such as imaging eye of a subject. For example, the imaging device may be used for retinal imaging for diagnosis and treatment purposes. To this end, a retinal image refers to a digital image of back of an eye of a subject. The retinal image may capture or show retina, optic disk, and blood vessels within the eye of the subject. Such retinal imaging may be used for eye exam or diagnosis of an eye disease.

Typically, the imaging device is hand-held for capturing a retinal image of an eye of a subject. Capturing the retinal image using the handheld imaging device may require great attentiveness. In particular, a user of the imaging device may have to undergo training in order to be able to capture a retinal image of an eye for examining of the eye. Further, the user may have to position the imaging device at a correct working distance and point the imaging device through a middle of pupil of the eye. The user may have to assess and adjust the imaging device to capture a precise retinal image, based on, for example, working distance, and lighting or brightness of environment. The user may hold the imaging device at a desired correct working distance in steady manner to capture the retinal image. Any deviation in the adjustment or any movement of the imaging device may yield imprecise or non-usable retinal image. In addition, difficulty in capturing the retinal image increases in cases where the pupil of the subject is small, or the subject may be non-cooperative.

In certain cases, a retinal image may be captured by directing near-infrared light into the retina. The user of the imaging device may then inspect a field of view of the imaging device and trigger capture when the correct working distance and positioning is achieved. This may reduce a need for dilation of the pupil of the subject. However, the user may still have to adjust brightness or lighting of the environment to capture accurate retinal image. Moreover, the user may still have to assess distribution of brightness across the field of view of the imaging device based on their experience. Such a standard may differ between technicians and hence the quality of the image thus captured may differ.

Capturing of retinal image may be simplified by triggering through automated mechanisms the capture of a retinal image at a correct working condition. In this regard, the retinal image captured at the correct working condition may be precise and have high resolution and brightness, thereby making them fit for medical use. Conventional auto-capture feature of an imaging device may rely on optics and detecting a trajectory of a light ray coming from retina for capturing a retinal image. In another example, an auto-capture feature may rely on image processing techniques to detect presence of retinal features in a field of view of the imaging device. However, operation and functioning of such auto-capture feature may not accurately or clearly show the retina. In particular, such auto-capture features may not ensure correct illumination of the retina of eye. As a result, it may be challenging for an examiner of the retinal image to derive accurate conclusion from the retinal image thereby rendering the auto-capture features for retinal imaging futile. Moreover, such auto-capture features may require additional processing devices thereby making the imaging device bulky and complex. This may hamper experience of the user using the imaging device and the subject.

Approaches for automated retinal image capturing, based on an analysis model, are described. In this context, the analysis model may be used for triggering capture of a retinal image based on visual attributes within a field of view of an imaging device. The analysis model employs machine learning techniques to provide mechanisms by way of which the visual attributes within field of view of the imaging device may be monitored. In such cases, the analysis model may be initially trained for assessing the visual attributes within the field of view of the imaging device and accordingly trigger automated retinal image capturing based on the assessment. Examples of such visual attributes include, but are not limited to, light distribution parameters, light intensity, brightness, contrast, sharpness, and shape.

In an example, an imaging device may record a plurality of target frames of a retina of a subject. In particular, the imaging device may be positioned in a path of light reflected from the retina for recording the plurality of target frames. As may be understood, the imaging device may collect light rays reflected from the retina and redirect them to a single point, i.e., focal point of the imaging device. In an example, the retina of an eye may be illuminated by near field-Infrared light.

To this end, the imaging device may record the plurality of target frames in a real-time mode of the imaging device. The plurality of target frames may be recorded before the actual capturing of the retinal image. For example, the plurality of target frames may be recorded by the imaging device during an adjustment of the imaging device with respect to an eye or the retina of the subject.

In operation, a system may receive the plurality of target frames from the imaging device. The system may analyze the plurality of target frames based on the analysis model. The analysis model may be initially trained based on a set of training images. In an example, the analysis model is trained based on visual attributes and annotations corresponding to the set of training images. In this regard, the analysis model may be considered as incorporating a set of confidence weights based on the visual attributes and the annotations for each of the set of training images. A confidence weight for a training image, in an example, may correspond to and may be determined based on a correlation between the visual attributes and the annotation for the training image.

In an example, the set of training images may correspond to infrared images of retina captured by imaging devices in the real-time mode. For example, the training of the analysis model may involve using the large set of training images by a training system where the analysis model may be trained. Moreover, the analysis model may be trained once before deploying the trained analysis model on the system.

Once trained, the set of confidence weights and the trained analysis model may be deployed on the system. The analysis model may then be utilized for analyzing the plurality of target frames recorded by the imaging device. For example, the analysis of the plurality of target frames are performed in real-time, i.e., at a time of recordal of the target frames. Based on the set of confidence weights and the plurality of target frames, the analysis model on the system may determine if any of the plurality of target frames corresponds to a correctly positioned image for capturing the retinal image. In an example, for a correctly positioned image, an imaging device may be positioned at a correct working distance and illuminated appropriately within the field of view of the imaging device.

On determining that a target frame corresponds to the correctly positioned image, the imaging device may be triggered to cause the imaging device to capture a retinal image of the retina of the subject. Such retinal image capturing is performed at correct working condition of the imaging device. For example, the correct working condition may be achieved when to the imaging device is held at a correct working distance, the field of view of the imaging device is appropriately illuminated, and visual attributes of the retinal image are correct.

The system for automated retinal images capturing described in the present subject matter employs machine learning-based analysis model for analyzing target frames for capturing the retinal image. Retinal image is thus captured by the imaging device at a correct working condition. The retinal image may be precise and have high resolution, thereby making them effective for medical use. In this manner, any noise or unwanted artefact is substantially reduced in the auto-captured retinal images. In an example, the techniques, i.e., the machine learning model, for automated retinal image capturing may be deployed on handheld systems, such as smartphones, digital camera, and the like. In such a manner, precise retinal images may be captured using existing imaging technology. In addition, the use of the machine learning model does not introduce complexity to the system thereby improving operability or ease of use of the system. Therefore, precise retinal images may be auto-captured without incurring any substantial increase in cost.

The present subject matter is further described with reference to the accompanying figures. Wherever possible, the same reference numerals are used in the figures and the following description to refer to the same or similar parts. It should be noted that the description and figures merely illustrate principles of the present subject matter. It is thus understood that various arrangements may be devised that, although not explicitly described or shown herein, encompass the principles of the present subject matter. Moreover, all statements herein reciting principles, aspects, and examples of the present subject matter, as well as specific examples thereof, are intended to encompass equivalents thereof.

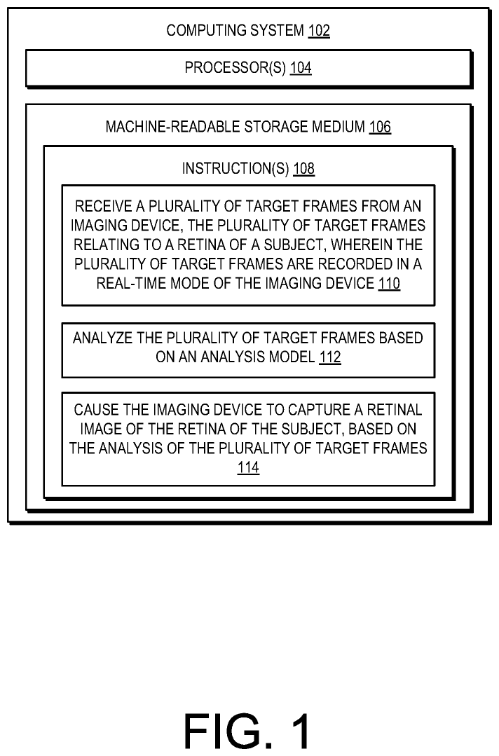

The manner in which the example systems are implemented are explained in detail with respect to FIGS. 1-7. While aspects of described system may be implemented in any number of different electronic devices, environments, and/or implementations, the examples are described in the context of the following example device(s). It is to be noted that drawings of the present subject matter shown here are for illustrative purposes and are not to be construed as limiting the scope of the subject matter claimed.

FIG. 1 illustrates an example system 102 for automated retinal image capturing, based on an analysis model, according to an example of the present subject matter. The system 102 includes a processor 104, and a machine-readable storage medium 106 which is coupled to, and accessible by, the processor 104. The system 102 may be a computing system, such as a storage array, server, desktop computer, a laptop computer, a smartphone, a distributed computing system, or the like. Although not depicted, the system 102 may include other components, such as interfaces to communicate over a network or with external storage or computing devices, display, input/output interfaces, operating systems, applications, data, and the like, which have not been described for brevity.

The processor 104 may be implemented as a dedicated processor, a shared processor, or a plurality of individual processors, some of which may be shared. The machine-readable storage medium 106 may be communicatively connected to the processor 104. Among other capabilities, the processor 104 may fetch and execute computer-readable instructions, including instructions 108, stored in the machine-readable storage medium 106. The machine-readable storage medium 106 may include non-transitory computer-readable medium including, for example, volatile memory such as RAM, or non-volatile memory such as EPROM, flash memory, and the like. The instructions 108 may be executed to determine occurrence of an anomaly in the target computing device, based on the analysis of the current operating parameters of the target computing device.

In an example, the processor 104 may fetch and execute instructions 108. For example, as a result of the execution of instructions 110, the system 102 may receive a plurality of target frames from an imaging device. The plurality of target frames may relate to a retina of a subject or a patient. The plurality of target frames may be captured in a real-time mode of the imaging device. In particular, the plurality of target frames may correspond to image frames of the retina of the subject from different position and angle of the imaging device. The plurality of target frames may correspond to image frames within a field of view of the imaging device, captured in real-time. In an example, such plurality of target frames may be stored temporarily in a memory, such as the machine-readable storage medium 106 of the system 102.

The plurality of target frames of the retina may be analyzed based on an analysis model, as a result of the execution of instructions 112. The analysis model may be a machine learning model. As mentioned previously, the analysis model may be trained prior to it being utilized for analyzing the plurality of target frames. In particular, the analysis model may be trained using a set of training images. In an example, the analysis model may be trained before use at a training system, wherein the training system may be different and distinct from the system 102.

Once trained, the system 102 may use the analysis model for analyzing the plurality of target frames of the retina received from the imaging device to determine whether the imaging device is suitably configured for capturing the retinal image. In particular, the system 102 may analyze the plurality of target frames in real-time, based on the trained analysis model. In this regard, the system 102 may analyze visual attributes corresponding to a target frame, based on or by using the analysis model. Based on the visual attributes for the target frame, the system 102 may ascertain if the target frame corresponds to correctly positioned image to determine whether the imaging device is suitably configured for capturing the retinal image.

With the plurality of target frames being analyzed in real-time, the system 102 may determine if at least one of the plurality of target frames corresponds to a correctly positioned image. Subsequently, instructions 114, when executed by the system 102 may cause the imaging device to capture the retinal image of the retina of the subject. The retinal image is captured instantaneous to a time of recordal of a target frame that corresponds to correctly positioned image. It may be noted that the correctly positioned image may correspond to an image that captures a retina appropriately, for example, from a correct working distance between an imaging device and the retina, while a field of view of the imaging device is appropriately illuminated, i.e., when there is no presence of any unwanted artefact. Based on the analysis of the plurality of target frames, the retinal image may be captured. The present approach is just one of the many other examples that may be used for automated retinal image capturing. Such other approaches may be used without limiting the scope of the present subject matter.

The above described techniques implemented as a result of the execution of the instructions 108 may be performed by different programmable entities. Such programmable entities may be implemented through computing systems, which may be implemented either on a stand-alone computing device, or multiple computing devices. As will be explained, various examples of the present subject matter are described in the context of a computing system for training a neural network-based model, and thereafter, utilizing the neural network model for automated retinal image capturing based on the analysis of the plurality of target frames. For example, such neural network-based model is the analysis model that is trained and deployed on the computing system for automated retinal image capturing. These and other examples are further described with respect to other figures.

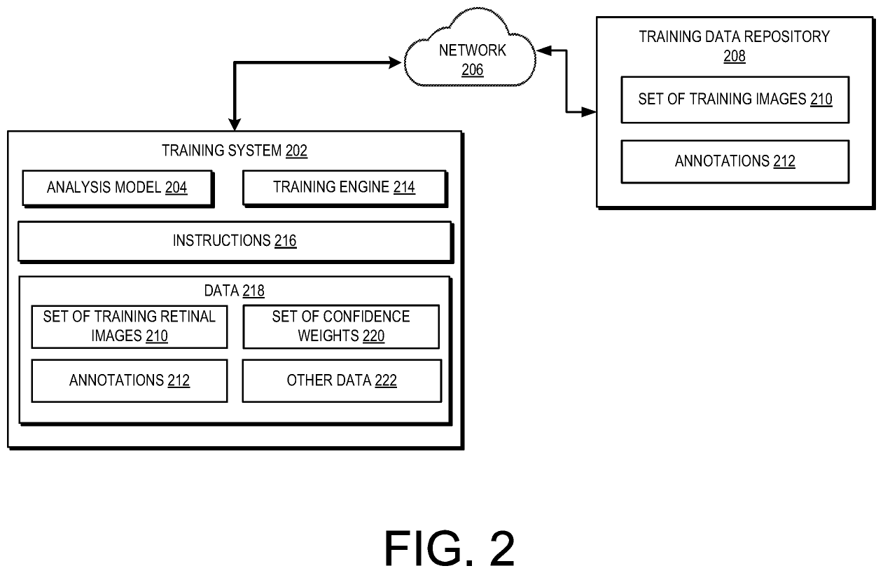

FIG. 2 illustrates a training system 202 for training an analysis model 204, according to an example of the present subject matter. The training system 202 comprises a processor and memory (not shown), for training the analysis model 204. In an example, the training system may be a processor-based system. Moreover, the training system 202 (referred to as the system 202) may be in communication, through a network 206, with a training data repository 208.

The network 206 may be a private network or a public network and may be implemented as a wired network, a wireless network, or a combination of a wired and wireless network. The network 206 may also include a collection of individual networks, interconnected with each other and functioning as a single large network, such as the Internet. Examples of such individual networks may include, but are not limited to, Global System for Mobile Communication (GSM) network, Universal Mobile Telecommunications System (UMTS) network, Personal Communications Service (PCS) network, Time Division Multiple Access (TDMA) network, Code Division Multiple Access (CDMA) network, Next Generation Network (NGN), Public Switched Telephone Network (PSTN), Long Term Evolution (LTE), and Integrated Services Digital Network (ISDN).

The training data repository 208 (referred to as repository 208) may be a machine-readable storage medium. The repository 208 may be coupled to, and accessible by, the system 202. The repository 208 may include non-transitory computer-readable medium including, for example, volatile memory such as RAM, or non-volatile memory such as EPROM, flash memory, and the like. The repository 208 may store training data, specifically, a set of training images 210 and annotations 212. The system 202 may use the set of training images 210 and the annotations 212 for training the analysis model 204.

Further, the system 202 may include a training engine 214. The training engine 214 (referred to as engine 214) or any other engine within the system 202, may be implemented as a combination of hardware and programming, for example, programmable instructions to implement a variety of functionalities. In examples described herein, such combinations of hardware and programming may be implemented in several different ways. For example, the programming for the engine 214 may be executable instructions, such as instructions 216. Such instructions may be stored on a non-transitory machine-readable storage medium which may be coupled either directly with the system 202 or indirectly (for example, through networked means). In an example, the engine 214 may include a processing resource, for example, either a single processor or a combination of multiple processors, to execute such instructions. In the present examples, the non-transitory machine-readable storage medium may store instructions, such as instructions 216, that when executed by the processing resource, implement engine 214. In other examples, the training engine 214 may be implemented as electronic circuitry.

The data 218 may include the set of training images 210 and the annotations 212 received by the system 202 from the repository 206. The data 218 further includes a set of confidence weights 220, and other data 222. The set of training images 210 may each have corresponding visual attributes and other parameters based on which the analysis model 204 may be trained. The system 202 may further include instructions 216 for training the analysis model 204 based on the visual attributes and other parameters of the set of training images 210. The visual attributes and other parameters may include data or values of different attributes pertaining to the set of training images 210. The visual attributes and other parameters may be derived by processing the set of training images 210 as a result of the execution of the instructions 216 or by an engine, such as the training engine 214.

In operation, the system 202 may receive the set of training images 210. As may be understood, each of the set of training images 210 may have attributes, such as the visual attributes and other parameters, relating to it. In an example, the system 202 may analyze the set of training images 210 to determine corresponding attributes.

Visual attributes for a training image may correspond to patterns of image segments that describes some characteristic properties, for the training image. In an example, such visual attributes may be any combination of appearance, shape, or the layout of segments within the training image. For example, the system 202 may process each of the set of training images 210 to identify corresponding visual attributes. As may be understood, the system 202 may perform such processing of the set of training images 210 per se or implicitly by its own.

Pursuant to the present subject matter, the visual attributes pertaining to a training image from the set of training images 210 may corresponds to visual attributes across a border of a retina of an eye within the training image, visual attributes at a center of the retina, visual attributes of pupil of the eye, visual attributes of optic disk, and visual attributes of blood vessels within the eye. Examples of the visual attributes pertaining to the set of training images 210 may include, but are not limited to, sharpness, resolution, brightness, light distribution parameters, light intensity, contrast, shape, size, color, texture, highlights, saturation, structure, and shadows. The visual attributes corresponding to a training image may relate to retina, optic disk, pupil and blood vessels of an eye captured within the training images.

It may be noted that a manner in which visual attributes occur within a training image may correspond to working conditions where the training image under consideration was captured. As the working conditions are varied, certain changes in the visual attributes may occur and may be present in the training image.

Each of the set of training images 210 may be associated with a corresponding annotation stored as annotations 212 based on which the analysis model 204 may be trained. In this regard, the annotations 212 may indicate the corresponding images from amongst the set of training images 210 as corresponding to a correctly positioned image or corresponding to an incorrectly positioned image. It may be understood that a correctly positioned image may be an intermediary image frame that may correspond to a correct position that may be fit for capture. On the other hand, an incorrectly positioned image may be an intermediary image frame that may correspond to an incorrect position that may not be fit for capture or use.

To this end, the annotations 212 may be assigned to the set of training images 210 for indicating the set of training images 210 as corresponding to the correctly positioned image that is fit for capture or the incorrectly positioned image that is unfit for capture. In an example, the annotations 212 may be a label, wherein the correctly positioned images from the set of training images 210 may be labeled as ‘correct’ and the incorrectly positioned images from the set of training images 210 may be labeled as ‘incorrect’.

In an example, the annotations 212 may also specify working distance which may be assigned to the set of training images 210. In particular, a working distance for a training image may correspond to a distance between an imaging device and an eye of which an image is captured. For example, such distance may be indicated as numerical value in micrometers, millimeters, centimeters, meters, and the like. In another example, the annotations 212 may also specify positioning information which may be assigned to the set of training images 210. Positioning information for a training image may correspond to an angle of an imaging device that captured the training image, such as an angle of the imaging device with respect to an eye or a retina.

In an example, such annotations 212 of the set of training images 210 as a correctly positioned imaged or an incorrectly positioned image, working distance, or positioning information for the set of training images 210 may be assigned manually or through processor-based automated means. Moreover, the set of training images 210 may be associated with corresponding visual attributes, such as the light distribution parameters, sharpness, brightness, contrast and other parameters corresponding to them.

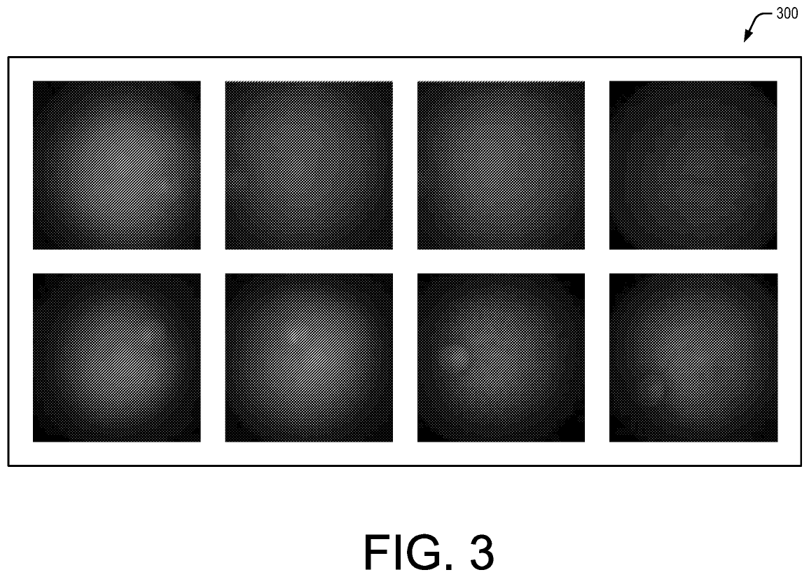

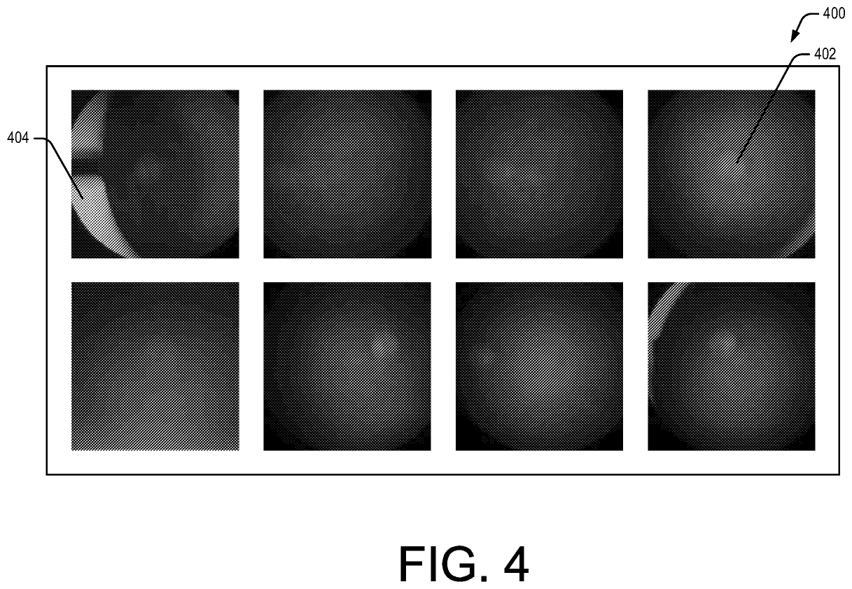

FIGS. 3 and 4 illustrate set of training images for training an analysis model, according to an example of the present subject matter. The set of training images (such as the set of training images 210) may be stored at a training data repository (such as the repository 206). In an example, each of the set of training images 210 may correspond to an infrared view of a retina. In this regard, a training image may be captured by illuminating an eye with IR light. Although the set of training images 210 for training the analysis model 204 are depicted as IR image frames, such depiction of the set of training images 210 should not be construed as a limitation. In other examples of the present subject matter, the set of training images may be retinal images, images captured by illuminating eye with white light, or images captured by illuminating eye with visible or IR light of any frequency.

Further, based on working condition, each of the set of training images 210 may correspond to a correctly positioned image or an incorrectly positioned image. It may be noted that a correctly positioned image may have correct working condition thereby rendering them fit for capture, while an incorrectly positioned image may have noise or incorrect working condition thereby rendering them unfit for capture.

FIG. 3 illustrates a set of correctly positioned images 300, as per an example. In particular, the set of correctly positioned images 300 may be captured under correct working condition. Such correct working condition may be achieved by, for example, appropriate illumination, appropriate working distance, appropriate angle, and the like. The set of correctly positioned images 300 is devoid of any noise, such as white spots, dark borders, and unwanted artefacts. Moreover, the set of correctly positioned images 300 captures an eye, specifically, a retina, a pupil, optic disk and blood vessels of the eye accurately. For example, the set of correctly positioned images 300 may be captured at a correct time, such as when a working distance between the eye and imaging device is correct, angle of the imaging device is correct, the eye or the retina is appropriately illuminated, and focused, and captured correctly.

FIG. 4 illustrates a set of incorrectly positioned images 400, as per an example. In particular, the set of incorrectly positioned images 400 may be captured under incorrect working condition. Such incorrect working condition may be due to, for example, improper illumination, incorrect working distance, improper angle, and the like. The set of incorrectly positioned images 400 may have noise, such as white spots 402, dark borders, and unwanted artefacts 404. Moreover, the set of incorrectly positioned images 400 captures an eye, specifically, a retina, a pupil, optic disk, and blood vessel of the eye incorrectly. For example, the set of incorrectly positioned images 400 may be captured at an incorrect time, such as when a working distance between the eye and imaging device is incorrect, angle of the imaging device is improper, the eye or the retina is not appropriately illuminated, i.e., image frames may be dark, blurry or out of focus.

Returning to FIG. 2, the system 202 may receive the set of training images 210 for training the analysis model 204. In this regard, the training engine 214 of the system 202 may train the analysis model 204 based on the set of training images 210. As would be understood, the analysis model 204 incorporates confidence weights 220 which may get further defined during the process of training as implemented by the training engine 214. The set of confidence weights 220 may refer to learnable parameters of a trainable machine learning model (like the analysis model 204) which are defined based on the set of training images 210.

In an example, the training engine 214 may identify the visual attributes for each of the set of training images 210. As mentioned previously, the visual attributes for the set of training images 210 may include, for example, light distribution parameters, sharpness, brightness, contrast, shape, structure, and so forth.

Thereafter, the training engine 214, using the analysis model 204, may correlate the visual attributes of the set of training images 210 with the corresponding annotations 212 associated with the set of training images 210. In an example, the training engine 214, using the analysis model 204, may correlate visual attributes of a training image with an annotation associated with the training image, wherein the annotation indicates if the training image corresponds to a correctly positioned image or an incorrectly positioned image. The above process may be repeated for each of the training images in the set of training images 210. Based on the correlation, the analysis model 204 may understand visual attributes associated with correctly positioned images and visual attributes associated with incorrectly positioned images. Based on the understanding, the set of confidence weights 220 of the analysis model 204 may be updated. In an example, the set of confidence weights 220 of the analysis model 204 may be updated based on each of the correlations between visual attributes of the set of training images 210 and corresponding annotations 212.

Although the present subject matter describes training of the analysis model 204 using labeled set of training images 210. However, such supervised training of the analysis model 204 should not be construed as a limitation. In other examples of the present subject matter, the analysis model 204 may be trained in an unsupervised manner, using unlabeled training dataset. In such a case, the analysis model 204 may analyze, for example, the visual attributes of the set of training images 210 to determine annotation corresponding to each of the set of training images 210. Accordingly, the set of confidence weights 220 may be updated.

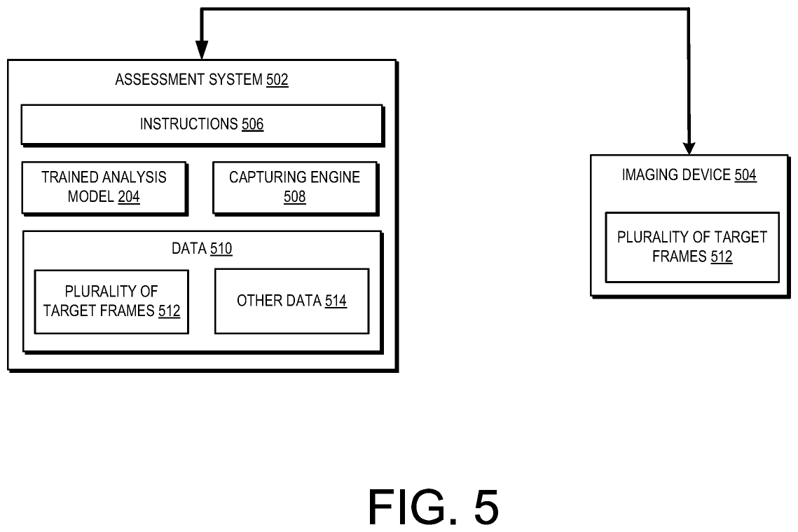

Once trained, the updated set of confidence weights 220 and the analysis model 204 may be deployed at an assessment system for automated retinal image capturing. The manner in which the analysis model triggers automated retinal image capturing is described in conjunction with FIG. 5.

FIG. 5 illustrates an assessment system 502 implementing a trained analysis model 204, according to an example of the present subject matter. The assessment system 502 (such as the system 102) comprises a processor and memory (not shown), for automated retinal image capturing. The assessment system 502 may further include the trained analysis model 204. Examples of the assessment system 502 include, but are not limited to, desktop computer, laptop computer, smartphone, digital camera, and camcorder.

Furthermore, the assessment system 502 may be operatively coupled to an imaging device 504. The imaging device 504 may be an electronic device capable of recording, storing, manipulating and transmitting a digital image. The imaging device 504 may include a number of components, such as an optical aperture, an imaging lens, a micro lens array, an imaging element, and a controller. The imaging lens of the imaging device 504 may collect light rays and redirect them to the imaging element of the imaging device 504. Due to the redirection of light rays to a single point, an image may be formed at the imaging element. The micro lens array of the imaging device 504 may enable a user to adjust, such as focus, zoom, and the like, the imaging device. Although, the imaging device 504 is depicted to be directly coupled to the assessment system 502, however, such depiction should not be construed as limiting. In certain embodiments of the present subject matter, the assessment system 502 may be coupled with the imaging device 504 over a network. The network, in an example, may be similar to the networks 208, as described in FIG. 2.

It may be noted that although the assessment system 502 is shown as distinct from the imaging device 504, the assessment system 502 and the imaging device 504 may be the same device, without deviating from the scope of the present subject matter. Pursuant to present example, the assessment system 502 may be a smartphone, wherein the smartphone includes the imaging device 504. In this regard, the imaging device 504 may be a camera of the smartphone.

The assessment system 502 may include instructions 506, that when executed, may implement the trained analysis model 204 for automated retinal image capturing. A retinal image may correspond to a digital picture of an eye of a subject. The digital picture may show retina, pupil, optic disk, and blood vessels of the eye of the subject. In particular, the retina may be a spot where light rays entering the eye may be redirected. As may be understood, an image may be formed at the retina of the eye due to the light rays entering the eye. Moreover, the optic disk may be a spot on the retina that holds optic nerve. For example, the retinal image of the retina may be examined to check health of the eye or for diagnosis of certain diseases, such as macular degeneration, glaucoma, retinal toxicity, and the like.

The assessment system 502 may further include a capturing engine 508. The capturing engine 508 may be implemented as a combination of hardware and programming, for example, programmable instructions to implement a variety of functionalities. The capturing engine 508 may be implemented in a similar manner as engine 214, as described in conjunction with FIG. 2. The capturing engine 508 may perform operations to facilitate retinal image capturing by the assessment system 502. The system 502 may further include data 510. The data 510 may include a plurality of target frames 512, and other data 514. The other data 514 may be any data that is generated or used by the assessment system 502 during its operation.

In operation, the imaging device 504 may record a plurality of target frames 512. The plurality of target frames 512 may relate to a retina of a subject. The plurality of target frames may be recorded in a real-time mode of the imaging device 504. The plurality of target frames 512 may correspond to image frames of a field of view of the imaging device 504 in real-time mode. For example, a camera application may be launched on a smartphone for activating the camera of the smartphone. The camera may be positioned with respect to an eye of the subject, for example, in front of the retina or above the retina, for recording the plurality of target frames 512. In such a case, the real-time mode may correspond to a live view of the camera of the smartphone, wherein the plurality of target frames 512 may correspond to image frames of the live view. For example, the eye of the subject may be illuminated with near Infrared light for recording the plurality of target frames 512 using the imaging device 504.

The assessment system 502 may execute instructions 506 to receive the plurality of target frames 512 recorded by the imaging device 504. It may be noted that such plurality of target frames 512 are not retinal images to be captured. The plurality of target frames 512 are intermediary image frames captured before capturing of the retinal image, for example, after launching of the camera application on the smartphone. Subsequently, the plurality of target frames 512 may be stored temporarily by the assessment system 502 for processing.

The assessment system 502 may execute instruction 506 to analyze the plurality of target frames 512 based on the trained analysis model 204. Before the use of the analysis model 204 at the assessment system 502, the analysis model 204 may be trained at a remote training system (such as the training system 202 described in conjunction with FIG. 2). As described above, the analysis model 204 may be trained based on a set of training images (such as the set of training images 210). It may be noted that the training images 210 are not retinal images, but intermediary image frames of retina of eye. During the training, the analysis model 204 may incorporate a set of confidence weights (such as the set of confidence weights 220) based on attributes of the set of training images 210. In an example, the attributes of the set of training images 210 may include, but are not limited to, visual attributes, such as light distribution parameters, brightness, contrast, sharpness, shape, structure and other parameters, and annotations. Once the analysis model 204 may be trained for use, it may be deployed on the assessment system 502.

In an example, the capturing engine 508 may execute instructions 506 to analyze each of the plurality of target frames 512, based on the trained analysis model 204. Based on the trained analysis model 204 incorporating the set of confidence weights 220, the plurality of target frames 512 may be analyzed. In particular, the capturing engine 508 may determine visual attributes for a target frame using the trained analysis model 204. In an example, the capturing engine 508 may determine visual attributes at a center of the target frame and border of the target frame, wherein the center of the target frame may depict pupil and the border of the target frame may depict border of the retina of the eye of the subject. In the present example, the capturing engine 508 may determine visual attributes corresponding to blood vessels of the eye of the subject.

In an example, based on visual attributes of a target frame, the capturing engine 508 may determine a working distance for the target frame, using the trained analysis model 204. The working distance may be a distance between the imaging device 504 and the retina of the subject, at a time when the target frame was captured. The capturing engine 508, using the trained analysis model 204, may further determine positioning information, such as angle, of the imaging device 504 with respect to the retina of the subject, at the time when the target frame was captured. For example, the analysis model 204 may correlate the determined visual attributes with working distance corresponding to the target frame.

The capturing engine 508, using the analysis model 204, may ascertain whether visual attributes for the target frame corresponds to correctly positioned image or incorrectly positioned image. In an example, the analysis model 204 may ascertain if the target frame corresponds to the correctly positioned image or incorrectly positioned image based on the visual attributes, working distance, and positioning information. In this manner, the plurality of target frames 512 may be analyzed. As would be understood, the plurality of target frames 512 may have variations in visual attributes, such as light distribution parameters, brightness, contrast, sharpness, and so forth. To this end, the trained analysis model 204 when implemented on the plurality of target frames 512 may correlate the visual attributes of the plurality of target frames 512 as correctly positioned image or incorrectly positioned image. Based on the analysis, at least one of the plurality of target frames may be identified, wherein the identified target frame may correspond to correctly positioned image.

As would be understood, a target frame identified as a correctly positioned image may record the retina of the subject correctly. For example, such target frame may not be blurred or out of focus, and may not include any noise, such as white spots, unwanted artefact, and dark border. Further, such target frame may be recorded from a correct working distance such that the imaging device 504 may be appropriately spaced from the eye of the subject and inclined at a correct angle with respect to the eye of the subject. As a result, the target frame may depict the retina of the subject in a pertinent manner.

The capturing engine 508 may then cause the imaging device 504 to capture the retinal image of the retina of the subject, based on the analysis of the plurality of target frames 512. The capturing engine 508 may trigger the imaging device 504 for automatically capturing the retinal image, based on the analysis model 204. On determining, using the analysis model 204, a target frame to correspond to a correctly positioned image, the capturing engine 508 may trigger the imaging device 504 to capture the retinal image. The retinal image is captured instantaneous to a time when the target frame corresponding to the correctly positioned image is recorded. The retinal image thus captured has correct working distance between the imaging device 504 and the retina, and the field of view of the imaging device 504 is appropriately illuminated, i.e., there is no presence of any unwanted artefact such as white spots at center or dark border.

In an example, when triggered, the imaging device 504 may cause to illuminate the eye of the subject with white light before capturing of the retinal image. For example, the imaging device 504 may illuminate the eye with white light using electronic flash. Once illuminated with white light, the imaging device 504 may capture the retinal image.

In certain cases, the capturing engine 508 may wait to identify at least two consecutive target frames that may correspond to correctly positioned image. In this regard, the capturing engine 508, using the analysis model 204, may determine if a first set of target frames from the plurality of target frames 512 correspond to correctly positioned images. For example, the first set of target frames from the plurality of target frames 512 comprise at least three target frames corresponding to the retina of the subject. Thereafter, the capturing engine 508, using the analysis model 204, may determine if the target frames within the first set of target frames are consecutive. For example, the capturing engine 508 may determine if the target frames within the first set of target frames are consecutive based on a time stamp associated with the first set of target frames. On determining the target frames within the first set of target frames to be consecutive, the capturing engine 508 may trigger the imaging device 504 for automatically capturing the retinal image.

Although the present example describes that the assessment system 502 receives the plurality of target frames 512 at a beginning of the operation. However, such implementation of the assessment system 502 should not be construed as a limitation. In other implementations of the present subject matter, receiving of the plurality of target frames 512 may be a continuous process. In this regard, at least one target frame may be received at the beginning of the processing or analysis, while other target frames from the plurality of target frames 512 may be received in due course of time. As a result, the plurality of target frames 512 may be analyzed as they are obtained.

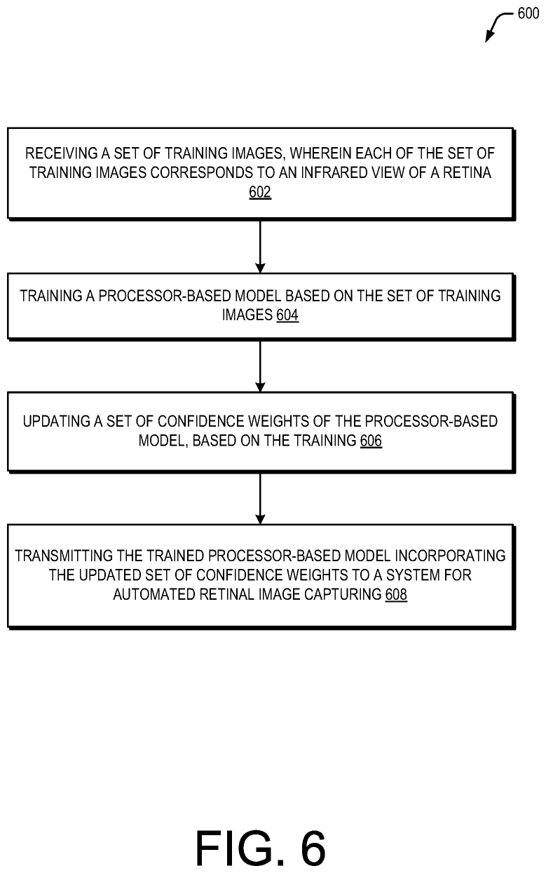

FIG. 6 illustrates an example method 600 for training a processor-based model for automated retinal image capturing, in accordance with example of the present subject matter. In an example, the processor-based model may be an analysis model (such as the analysis model 204). The order in which the above-mentioned method 600 is described is not intended to be construed as a limitation, and some of the described method blocks may be combined in a different order to implement the method, or alternative method.

Furthermore, the above-mentioned method 600 may be implemented in a suitable hardware, computer-readable instructions, or combination thereof. The steps of such method may be performed by either a system under the instruction of machine executable instructions stored on a non-transitory computer readable medium or by dedicated hardware circuits, microcontrollers, or logic circuits. For example, the method 600 may be performed by a training system 202. Herein, some examples are also intended to cover non-transitory computer readable medium, for example, digital data storage media, which are computer readable and encode computer-executable instructions, where said instructions perform some or all the steps of the above-mentioned method.

At block 602, a set of training images are received. The set of training images (such as the set of training images 210) may correspond to intermediate image frames of retina. In an example, the training system 202 may receive the set of training images 210 from a repository source, such as the training data repository 206. In another example, a user may upload the set of training images 210 on the training system 202.

In an example, each of the set of training images 210 may have corresponding attributes. Such attributes may include, for example, visual attributes, and other parameters. In addition, the set of training images 210 may be associated with a corresponding annotation (such as an annotation form the annotations 212) based on which the analysis model 204 may be trained. For example, an annotation for a training image may indicate the training image as a correctly positioned image or an incorrectly positioned image.

At block 604, the processor-based model is trained based on the set of training images. The system 202 may include the analysis model 204. In an example, a training engine (such as the training engine 214) may execute instructions for training the analysis model 204, based on the set of training images 210. Attributes of the set of training images 210, such as the visual attributes and other parameters, and annotations 212 associated with the set of training images 210 may be considered for training of the analysis model 204. For example, the visual attributes of each of the set of training images 210 may be correlated with corresponding annotation from the annotations 212. Based on the correlation, the analysis model 204 may be trained to identify visual attributes pertaining to a correctly positioned image and visual attributes pertaining to an incorrectly positioned image.

At block 606, a set of confidence weights of the processor-based model may be updated, based on the training. In an example, the analysis model 204 incorporates the set of confidence weights 220. For example, the set of confidence weights 220 may represent correlations between the visual attributes of the set of training images 210 and corresponding annotation 212. During the training of the analysis model 204, the set of confidence weights 220 incorporated in the analysis model 204 may be refined and updated by the training engine 212.

At block 608, the trained processor-based model incorporating the updated set of confidence weights may be transmitted to an assessment system for automated retinal image capturing. To this end, the trained analysis model 204 incorporating the updated set of confidence weights 220 may be transmitted to an assessment system (such as the assessment system 502). At the assessment system 502, the analysis model 204 may be executed by an engine, such as the capturing engine 508, to analyze a plurality of target frames 512. The capturing engine 508 may determine correctly positioned target frames from the plurality of target frames 512, using the trained analysis model 204. As a result, the capturing engine 508 may trigger an imaging device 504 for automated capturing of a retinal image of a retina of a subject.

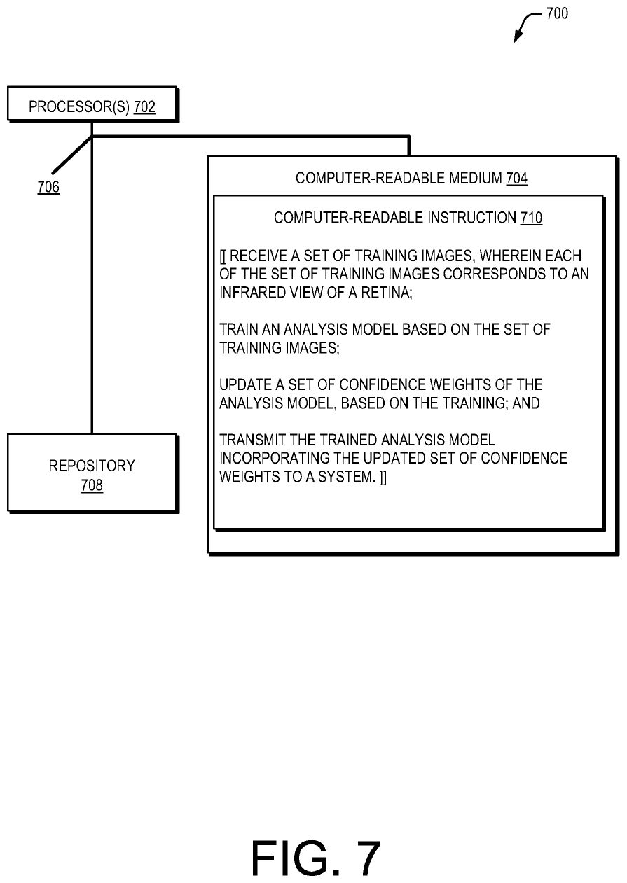

FIG. 7 illustrates a computing environment 700 implementing a non-transitory computer readable medium for training an analysis model for automated retinal image capturing. In an example, the computing environment 700 includes processor(s) 702 communicatively coupled to a non-transitory computer readable medium 704 through a communication link 708. In an example, the processor(s) 702 may have one or more processing resources for fetching and executing computer-readable instructions from the non-transitory computer readable medium 704. The processor(s) 702 and the non-transitory computer readable medium 704 may be implemented, for example, in system 202 (as has been described in conjunction with the FIG. 2).

The non-transitory computer readable medium 704 may be, for example, an internal memory device or an external memory device. In an example implementation, the communication link 706 may be a network communication link. The processor(s) 702 and the non-transitory computer readable medium 704 may be communicatively coupled to a training data repository 708 (similar to the training data repository 206) over the network. The processor(s) 702 and the non-transitory computer readable medium 704 may also be communicatively coupled to an assessment system (such as the assessment system 502) over the network.

In an example implementation, the non-transitory computer readable medium 704 includes a set of computer readable instructions 710 which may be accessed by the processor(s) 702 through the communication link 706. Referring to FIG. 7, in an example, the non-transitory computer readable medium 704 includes instructions 710 that cause the processor(s) 702 to receive a set of training images, such as the set of training images 210. In an example, the set of training images 210 may be intermediary image frames that correspond to infrared view of retina captured by imaging device(s). The instructions 710 may cause the processor(s) 702 to train the analysis model 204 based on the set of training images 210. In an example, the set of training images 210 may have corresponding visual attributes, such as light distribution parameters, brightness, contrast, sharpness, and other parameters, and may be associated with a corresponding annotation. For the training of the analysis model 204, the visual attributes for each of the set of training images 210 may be correlated with corresponding annotation and considered. In an example, visual attributes corresponding to correctly positioned images and visual attributes corresponding to incorrectly positioned images may be determined from the set of training images 210.

During the training of the analysis model 204, the instructions 710 may further cause the processor(s) 702 to update a set of confidence weights of the analysis model 204. The analysis model 204 may incorporate the set of confidence weights 220. For example, a training engine (such as the training engine 214) while training the analysis model, may cause periodic updating of the set of confidence weights 220. As may be noted, updated value of the set of confidence weights 220 may be refined or improved as compared to old value of the set of confidence weights 220.

After the training of the analysis model 204, the instructions 710 may be executed which cause the processor(s) 702 to transmit the trained analysis model 204 incorporating the updated set of confidence weights 220 to a system. The analysis model 204 may also incorporate visual attributes of correctly positioned images and incorrectly positioned images. The trained analysis model 204 transmitted to the system (such as the assessment system 502) may be executed by a capturing engine (such as the capturing engine 508). The capturing engine 508 may analyze a plurality of target frames corresponding to a retina of a subject, based on the trained analysis model 204. Based on the analysis, the capturing engine 508 may ascertain if a target frame from the plurality of target frames corresponds to a correctly positioned image. On ascertaining that the target frame corresponds to correctly positioned image, the capturing engine 508 may cause an imaging device (such as the imaging device 504) to capture a retinal image of the retina of the subject.

Although examples for the present disclosure have been described in language specific to structural features and/or methods, it is to be understood that the appended claims are not necessarily limited to the specific features or methods described. Rather, the specific features and methods are disclosed and explained as examples of the present disclosure.

1. A system for automated retinal image capturing, the system comprising:
a processor;
a machine-readable storage medium comprising instructions executable by the processor to:
receive a plurality of target frames from an imaging device, the plurality of target frames relating to a retina of a subject, wherein the plurality of target frames are recorded in a real-time mode of the imaging device;
analyze the plurality of target frames based on an analysis model, wherein:
the analysis model is trained based on a set of training images, the analysis model incorporating a set of confidence weights, wherein the set of confidence weights is based on visual attributes and annotation for each of the set of training images; and
cause the imaging device to capture a retinal image of the retina of the subject, based on the analysis of the plurality of target frames.

2. The system as claimed in claim 1, wherein visual attributes pertaining to a training image corresponds to visual attributes across a border of a retina within the training image, visual attributes at a center of the retina, visual attributes of pupil of an eye within the training image, and visual attributes of blood vessels within the eye.

3. The system as claimed in claim 1, wherein visual attributes for a training image comprises one of light distribution parameters, sharpness, contrast, and brightness, within the training image.

4. The system as claimed in claim 1, wherein an annotation for a training image indicates a working distance between an imaging device and a retina within the training image, and a positioning information of the imaging device.

5. The system as claimed in claim 1, wherein an annotation for a training image indicates the training image as corresponding to a correctly positioned image that is fit for capture or corresponding to an incorrectly positioned image that is unfit for capture.

6. The system as claimed in claim 1, wherein the analysis of the plurality of target frames comprises:
determining visual attributes for each of the plurality of target frames;
determining whether at least one of the plurality of target frames corresponds to a correctly positioned image that is fit for capture, based on the corresponding visual attributes; and
causing the imaging device to capture the retinal image of the retina based on the determination.

7. The system as claimed in claim 6, wherein the analysis of the plurality of target frames further comprise:
determining whether a first set of target frames from the plurality of target frames correspond to correctly positioned images;
determining whether the target frames within the first set of target frames are consecutive; and
causing the imaging device to capture the retinal image of the retina based on the determination.

8. The system as claimed in claim 7, wherein the first set of target frames from the plurality of target frames comprise at least three target frames corresponding to the retina of the subject.

9. The system as claimed in claim 1, wherein the retina is illuminated with near-Infrared light for capturing the plurality of target frames using the imaging device.

10. The system as claimed in claim 6, wherein on determining that at least one of the plurality of target frames corresponds to a correctly positioned image that is fit for capture, the retina is illuminated with white light to cause capturing of the retinal image.

11. A method for training a processor-based model for automated retinal image capturing, the method comprising:
receiving a set of training images, wherein each of the set of training images corresponds to an infrared view of a retina;
training the processor-based model based on the set of training images;
updating a set of confidence weights of the processor-based model, based on the training; and
transmitting the trained processor-based model incorporating the updated set of confidence weights to a system for automated retinal image capturing.

12. The method as claimed in claim 11, wherein training the processor-based model comprises:
determining visual attributes for each of the set of training images; and
correlating the visual attributes for each of the set of training images with a corresponding annotation.

13. The method as claimed in claim 12, wherein an annotation for a training image indicates the training image as corresponding to a correctly positioned image that is fit for capture or an incorrectly positioned image that is unfit for capture.

14. The method as claimed in claim 11, wherein the processor-based model is trained at a training system.

15. The method as claimed in claim 11, wherein the processor-based model, once trained, is deployed on an assessment system that is operatively coupled to an imaging device.

16. The method as claimed in claim 15, wherein the assessment system is to:
receive a plurality of target frames from the imaging device, the plurality of target frames relating to a retina of a subject, wherein the plurality of target frames are recorded in a real-time mode of the imaging device;
analyze the plurality of target frames, based on the trained processor-based model; and
cause the imaging device to capture a retinal image of the retina of the subject, based on the analysis of the plurality of target frames.

17. A non-transitory computer-readable medium comprising instructions, the instructions being executable by a processing resource to:
receive a set of training images, wherein each of the set of training images corresponds to an infrared view of a retina;
train an analysis model based on the set of training images;
update a set of confidence weights of the analysis model, based on the training; and
transmit the trained analysis model incorporating the updated set of confidence weights to a system.

18. The non-transitory computer-readable medium as claimed in claim 17, comprising instructions executable by a processing resource to:
receive, at the system, the trained analysis model;
receive, at the system, a plurality of target frames relating to a retina of a subject from an imaging device, the plurality of target frames recorded in a real-time mode of the imaging device;
analyze, at the system based on the trained analysis model, the plurality of target frames to determine if at least one of the plurality of target frames correspond to correctly positioned image that is fit for capture; and
cause, by the system, the imaging device to capture a retinal image of the retina of the subject, based on the determination.
